(19) United States
(12) Patent Application Publication
(10) Pub. No.: US 2003/0158087 A1
(43) Pub. Date: Aug. 21, 2003

(54) Novel use of fulmonary surfactant for the prophylaxis or early treatment if acute fulmonary diseases

(76) Inventors: Dietrich Hafner, Konstanz (DE); Andreas Keller, Reichenau (DE); Frank Rathgeb, Konstanz (DE); Peter Schaffer, Radolfzell (DE); Wilhelm Wurst, Konstanz (DE); Christoph Karl, Konstanz (DE)

Correspondence Address: NATH & ASSOCIATES PLLC, 1030 FIFTEENTH STREET, N.W., SIXTH FLOOR, WASHINGTON, DC, 20005, US

(21) Appl. No.: 10/240,324

(22) PCT Filed: Apr. 12, 2001
(86) PCT No.: PCT/EP01/04223

(30) Foreign Application Priority Data
Apr. 12, 2000 (EP) 00107858.3

(51) Int. Cl. A61K 38/17

(52) U.S. Cl. 514/2

(57) ABSTRACT

The invention describes the novel use of pulmonary surfactant preparations for the prophylaxis or early treatment of acute pulmonary diseases.

### TECHNICAL FIELD OF THE INVENTION

The invention relates to the novel use of pulmonary surfactant preparations for the prophylaxis or early treatment of acute pulmonary diseases.

### PRIOR ART

ARDS (Adult Respiratory Distress Syndrome) is a descriptive expression which is applied to a large number of acute, diffuse infiltrative pulmonary lesions of differing etiology if they are associated with a severe gas exchange disorder (in particular arterial hypoxemia). (G. R. Bernard et al.: Report of the American-European consensus conference on ARDS: definitions, mechanisms, relevant outcomes and clinical trial coordination; Intensive Care Medicine, 1994, 20:225-232). The expression ARDS Is used for IRDS (Infant Respiratory Distress Syndrome) because of the numerous common clinical and pathological features. If, in the case of IRDS, the lung surfactant deficiency caused by premature birth is predominant, then in the case of ARDS a lung surfactant malfunction is caused by the disease of the lung based on differing etiologies. Triggering causes for an ALI (Acute Lung Injury) including ARDS can, for example, be (cited in accordance with Harrison's Principles of Internal Medicine 10th Ed. 1983 McGraw-Hill Int. Book Comp.) diffuse pulmonary infections (e.g. due to viruses, bacteria, fungi), aspiration of, for example, gastric juice or in the case of near-drowning, inhalation of toxins or irritants (e.g. chlorine gas, nitrogen oxides, smoke), direct or indirect trauma (e.g. multiple fractures or pulmonary contusion), systemic reactions to inflammations outside the lung (e.g. hemorrhagic pancreatitis, gram-negative septicemia), transfusions of high blood volumes or alternatively after cardiopulmonary bypass.

The therapy of ARDS present mainly consists in the earliest possible application of different forms of ventilation [e.g. PEEP (positive end-expiratory pressure), raising of the oxygen concentration of the respiratory air, SIMV (Synchronized Intermittent Mandatory Ventilation; Harrison's Principles of Internal Medicine 10th Ed. 1983 McGraw-Hill Int. Book Comp)] up to extracorporeal membrane oxygenation (ECMO; Zapol and Lemaire Adult Respiratory Distress Syndrome, Marcel Dekker Inc. 1991). The specific use of various ventilation techniques has only led to a small lowering of mortality and includes the risk of setting in motion a vicious circle. By ventilation with pressure and high FIO₂ (Fraction of Inspired Oxygen; proportion of oxygen in the respiratory air), the lungs themselves can be damaged and as a result of this even higher pressures and higher FiO₂ may be required in order to obtain an adequate oxygenation of the blood. For many years, it has proven suitable to treat IRDS by introducing pulmonary surfactant preparations into the lungs of the children concerned. It is known from pilot studies that pulmonary surfactant preparations are also clinically active in ALI including ARDS (survey, for example, B. Lachmann, D. Gommers and E. P. Eijking: Exogenous surfactant therapy in adults, Atemw.-Lungenkrkh. 1993, 19:581-91; D. Walmrath et al.: Bronchoscopic surfactant administration in patients with severe adult respiratory distress syndrome and sepsis, Am. J. Respir. Crit. Care Med. 1996, 154:57-62; T. J. Gregory et al.: Bovine surfactant therapy for patients with acute respiratory distress syndrome, Am. J. Respir. Crit. Care Med. 1997, 155:1309-15).

Surfactant abnormalities of differing severity are also reported for a number of other disease conditions, for example in obstructive pulmonary disorders such as asthma, bronchiolitis, COPD (Chronic Obstructive Pulmonary Disease) and after lung transplantation or alternatively after cardiopulmonary bypass (survey, see, for example, M. Griese Eur. Respir. J. 1999; 13: 1455-1467). Macnaughton et al. (Chest 1994; 105: 421425) and DoCampo et al. (Lancet 1994; 343: 482) describe the administration of exogenous surfactant after cardiopulmonary bypass. McBrien et al. (Lancet 1993; 342:1485-1486) and Suzuki et al. (Eur. J. Pediatr. 1996; 155: 383-384) describe the administration of surfactant after near-drowning. Strüber et al. (Cardiovasc. Surg. 1995; 110: 563-564) describe the administration of surfactant after lung transplantation.

### DESCRIPTION OF THE INVENTION

The object of the present invention is the provision of treatment methods and medicaments for the prophylaxis or early treatment of acute pulmonary diseases. Surprisingly, it has now been found that pulmonary surfactant preparations, in particular those which contain recombinant surfactant proteins, are suitable for the prophylaxis or early treatment of acute pulmonary diseases in mammals. As a result of the prophylactic administration of pulmonary surfactant in patients with the risk of an acute lung disease, the risk of an acute lung disease can be lowered. Thus, for example, by the treatment of ALI or ARDS patients at risk with pulmonary surfactant preparations the formation of ALI or ARDS can be prevented or the intensity of ALI or ARDS can be attenuated and thus the mortality rate associated with ALI or ARDS can be lowered. In particular, a progression to ARDS can be prevented even in patients with ALI or the intensity of ARDS can be attenuated. The stay of patients in intensive care units can be shortened and thus costs can be saved. Furthermore, in patients who are ventilated, it is possible by the administration of pulmonary surfactant to avoid side effects of ventilation, for example the risk of a nosocomial infection or pneumonia for the patients can be lowered or prevented.

In a first aspect, the invention therefore relates to the use of a pulmonary surfactant preparation for the production of medicaments for the prophylaxis or early treatment of acute pulmonary diseases in mammals.

Exemplary acute lung diseases according to the invention are ALI, ARDS, acute respiratory insufficiency, pneumonias (in particular ventilation-induced pneumonias), nosocomial infections or SIRS (systemic inflammatory response syndrome) associated with ALI.

According to the invention, the mammals are preferably humans, preferably patients in which the risk of the development of an acute lung disease exists. In particular, these are patients at risk of ALI or ARDS, patients in which the risk of acute respiratory insufficiency exists, patients in which the risk of pneumonia exists, patients in which the risk of a nosocomial infection exists, patients with hypothermia or patients with SIRS associated with ALI. By way of example, patients selected from the following patient groups may be mentioned: patients before, during or after intervention on the open thorax, patients who are ventilated, patients with pulmonary intoxication, patients with trauma, patients with sepsis, patients with pneumonia or those in which the risk of pneumonia exists, patients with a nosocomial infection or in which the risk of a nosocomial infection exists, patients with hypothermia and patients with SIRS associated with ALI.

According to the invention, prophylaxis of acute lung diseases in mammals is understood as meaning the complete or partial prevention (i.e. attenuation) of an acute lung disease, in particular in mammals which are not yet suffering from the acute lung disease or whose predisposing factors allow such a development to be assumed.

According to the invention, early treatment of acute lung diseases in mammals is understood as meaning the treatment of mammals which are in the stage of development of an acute lung disease.

Natural pulmonary surfactant has surface-active properties; it reduces, for example, the surface tension in the alveoli. A simple and rapid in vitro test with which the surface activity of pulmonary surfactant can be determined is, for example, the so-called Wilhelmy balance [Goerke, J. Biochim. Biophys. Acta, 344: 241-261 (1974), King R. J. and Clements J. A., Am. J. Physicol. 223: 715-726 (1972)]. This method gives information on the pulmonary surfactant quality, measured as the action of a pulmonary surfactant of achieving a surface tension of almost zero mN/m. Another measuring device for determining the surface activity of pulmonary surfactant is the pulsating bubble surfactometer [Possmayer F., Yu S. and Weber M., Prog. Resp. Res., Ed. v. Wichert, Vol. 18: 112-120 (1984)].

The activity of a pulmonary surfactant preparation can also be determined by means of in vivo tests, for example as described by Häfner et al. (D. Häfner et al.: Effects of rSP-C surfactant on oxygenation and histology in a rat lung lavage model of acute lung injury. Am. J. Respir. Crit. Care Med. 1998, 158: 270-278). By the measurement of, for example, the pulmonary compliance, the blood gas exchange or the ventilation pressures needed, it is possible to obtain information on the activity of a pulmonary surfactant.

Pulmonary surfactant preparation is understood according to the invention as meaning the numerous known compositions and their modifications which have the function of natural pulmonary surfactant. In this case, preferred compositions are those which, for example, have activity in the tests described above. Particularly preferred compositions are those which exhibit increased activity in such a test in comparison with natural, in particular human, pulmonary surfactant. In this context, these can be compositions which only contain phospholipids, but also compositions which, apart from the phospholipids, inter alia additionally contain pulmonary surfactant protein. Preferred phospholipids according to the invention are dipalmitoylphosphatidylcholine (DPPC), palmitoyloleylphosphatidylglycerol (POPG) and/or phosphatidylglycerol (PG). Particularly preferably, the phospholipids are mixtures of various phospholipids, in particular mixtures of dipalmitoylphosphatidylcholine (DPPC) and palmitoyloleylphosphatidylglycerol (POPG), preferably in the ratio from 7 to 3 to 3 to 7. Commercial products which may be mentioned are Curosurf® (Serono, Pharma GmbH, Unterschleiβheim), a natural surfactant from homogenized porcine lungs, Survanta® (Abbott GmbH, Wiesbaden) and Alveofact® (Boehringer Ingelheim), both extracts of bovine lungs, as well as Exosurf® (Glaxo Wellcome), a synthetic phospholipid containing excipients. Suitable pulmonary surfactant proteins are both the proteins obtained from natural sources, such as pulmonary lavage or extraction from amniotic fluid, and the proteins prepared by genetic engineering or chemical synthesis. According to the invention, in particular the pulmonary surfactant proteins designated by SP-B and SP-C and their modified derivatives are of interest. The amino acid sequences of these pulmonary surfactant proteins, their isolation or preparation by genetic engineering are known (e.g. from WO 86/03408, EP-A0251449, WO-89/04326, WO 87/06943, WO 88/03170, WO 91/00871, EP-A0368823 and EP-A-0 348 967). Modified derivatives of the pulmonary surfactant proteins designated by SP-C, which differ from human SP-C by the replacement of a few amino acids, are described, for example, in WO 91/18015 and WO 95/32992. Particularly to be emphasized in this connection are the recombinant SP-C derivatives which are disclosed in WO 95/32992, in particular those which differ from human SP-C in positions 4 and 5 by the replacement of cysteine by phenylalanine and in position 32 by the replacement of methionine by isoleucine [designated below as rSP-C (FF/I) or lusupultide (INN)]. Modified derivatives of pulmonary surfactant proteins are also understood as meaning those proteins which have a completely originally designed amino acid sequence with respect to their pulmonary surfactant properties, such as are described in EP-A 0593094 and WO 92/22315. Preferably, the polypeptide KL4 (INN: sinapultide) may be mentioned in this connection. The name pulmonary surfactant protein, according to the invention, also comprises mixtures of different pulmonary surfactant proteins. In EP-B 0100910, EP-A 0110498, EP-B 0119056, EP-B 0145005 and EP-B 0286011, phospholipid compositions with and without pulmonary surfactant proteins are described which are likewise suitable as components of the preparations.

As further constituents which can be present in pulmonary surfactant preparations, fatty acids such as palmitic acid may be mentioned. The pulmonary surfactant preparations can also contain electrolytes such as calcium, magnesium and/or sodium salts (for example calcium chloride, sodium chloride and/or sodium hydrogencarbonate) in order to establish an advantageous viscosity. Preferred preparations according to the invention contain 80 to 95% by weight of phospholipids, 0.5 to 3.0% by weight of pulmonary surfactant proteins, 3 to 15% by weight of fatty acid, preferably palmitic acid, and 0 to 3% by weight of calcium chloride.

The pulmonary surfactant preparations are prepared by processes known per se and familiar to the person skilled in the art, for example as described in WO 95/32992. According to the invention, the pulmonary surfactant preparations are preferably lyophilized and in particular spray-dried pulmonary surfactant preparations. Lyophilized preparations are disclosed, for example, in WO 97/35882, WO 91/00871 and DE 3229179. WO 97/26863 describes a process for the preparation of powdered pulmonary surfactant preparations by spray drying. According to the invention, preparations prepared in this way are preferred.

The patients having interventions on the open thorax can be, according to the invention, patients in which an intervention is carried out on the heart, such as a bypass operation or a heart valve operation. Furthermore, they can be patients in which an intervention is carried out on the lung, such as a lung transplantation or a pneumonectomy. In connection with the treatment of patients with lung transplantations, according to the invention a preliminary treatment of the organ to be transplanted with pulmonary surfactant preparation is preferably carried out before the transplantation, in particular before the storage of the transplant, particularly preferably before removal of the transplant from the organ donor. Novick et al. (Evaluation of Surfactant Treatment Strategies after Prolonged Graft Storage in Lung Transplantation; Am. J. Respir. Crit. Care Med. 1996, Vol. 154, 98-104) describe surfactant treatment strategies in connection with storage of lung transplants. Preferably, the patient who receives the organ donation is pretreated before transplantation with pulmonary surfactant preparation. After transplantation has taken place, further treatment of the patient with the pulmonary surfactant preparation can then be carried out.

According to the invention, ventilation of a patient is understood as meaning ventilation of the lungs which is brought about by aids or artificial respiration. An exemplary aid for ventilation which may be mentioned is the respirator, where different forms of ventilation known to the person skilled in the art can be used. Patients who are ventilated are, in particular, patients where spontaneous respiration is absent or insufficient. By way of example, patients having respiratory insufficiency, patients having central or peripheral respiratory paralysis, patients having ventilation under anesthesia, patients having long-term ventilation in intensive medicine and patients who are ventilated in the course of resuscitation. The ventilation of patients contains the risk of damaging the lung (Ventilation-Induced Lung Injury; VILI) and setting a vicious circle in motion. As a result of ventilation using pressure and a high FiO₂ (Fraction of Inspired Oxygen; proportion of oxygen in the respiratory air), the lung itself can be damaged and this can have the result that even higher pressures and higher FiO₂ are needed in order to obtain adequate oxygenation of the blood. At the same time, in the case of mechanically ventilated patients an increased risk of pneumonias and nosocomial infections exists (Michael J. Richards et al;. Critical Care Medicine 1999; 27:887-892). The prophylactic treatment of mechanically ventilated patients, in particular of those in which no ALI or ARDS is yet present, with pulmonary surfactant preparations can therefore lead to earlier weaning from the mechanical ventilation, lower the risk of setting a vicious circle in motion and additionally also decrease the risk of a nosocomial infection or pneumonia in such patients. The invention further also relates to a procedure for the mechanical ventilation of a patient, pulmonary surfactant preparation being administered to the mechanically ventilated patient. According to the invention, the patient is preferably a patient who has not yet developed ALI or ARDS.

Patients having pulmonary intoxication can be, for example, patients having pulmonary intoxication as a result of a bone marrow transplantation (toxic lung injury after bone marrow transplantation), or patients having a pulmonary intoxication which was caused by toxic gases.

The patients having trauma are, according to the invention, in particular patients having thoracic, cranial or cerebral trauma or those having multiple traumas.

Patients having hypothermia are in particular patients having a lowered body temperature in the case of collapse, hypothyroidism, cachexia, accidental hypothermia due to exposure to cold (e.g. in mountain and drowning accidents) and controlled hypothermia as is used, for example, in open heart surgery, in neurosurgery and in transplantations.

A further subject of the invention is a process for the prophylaxis or early treatment of acute pulmonary diseases in mammals, including humans, in particular of those in which the risk of the development of ARDS or ALI exists. The process is characterized in that a therapeutically efficacious and pharmacologically tolerable amount of a pulmonary surfactant preparation is administered to the mammal concerned. The dosage of the pulmonary surfactant preparations is carried out in the order of magnitude customary for pulmonary surfactant preparations. According to the invention, the pulmonary surfactant preparation in the case of the prophylaxis of ALI or ARDS Is preferably administered to the patient before the development of an ALI or ARDS condition.

The pulmonary surfactant preparation is administered in a manner known to the person skilled in the art, preferably by intratracheal instillation (infusion or bolus) of a pulmonary surfactant solution or suspension or in the form of an atomization of a pulmonary surfactant solution or suspension or by atomization of pulmonary surfactant powder. Preferably, the preparations according to the invention for administration are dissolved or suspended in a suitable solvent or resuspension medium, in particular if the preparations are present in lyophilized or spray-dried form. Preferably, the suitable resuspension medium is a physiological saline solution. It has proven advantageous to administer suspensions or solutions of the preparations according to the invention which contain 12.5 to 100 mg of phospholipids per ml of suspension. Preferably, the preparations according to the invention are administered per application in such an amount that the amount of phospholipids is between 12.5 and 200 mg per kilogram of body weight. As a rule, administration is carried out 1 to 3 times daily over a period of 1 to 7 days. A process is preferred in which the pulmonary surfactant solution employed contains 0.5 to 2.0 mg of rSP-C (FF/1) per ml of solvent. Particular mention may be made of a process in which the pulmonary surfactant solution employed contains 0.75 to 1.5 mg of rSP-C (FF/1) per ml of solvent. If desired, before the administration of the preparations according to the invention a bronchoalveolar lavage, preferably with dilute pulmonary surfactant preparation, can be carried out. Such a procedure is described, for example, in Gommers et al. [Bronchoalveolar lavage with a diluted surfactant suspension prior to surfactant instillation improves the effectiveness of surfactant therapy in experimental acute respiratory distress syndrome (ARDS), Intensive Care Med. 1998, 24:494-500] and in WO 98149191.

A further subject of the invention is a commercial product comprising a customary secondary packaging, a primary packaging comprising a pharmaceutical preparation (for example an ampoule) and, if desired, a pack insert, the pharmaceutical preparation being suitable for the prophylaxis or early treatment of acute pulmonary diseases in mammals and reference being made on the secondary packaging or on the pack insert of the commercial product to the suitability of the pharmaceutical preparation for the prophylaxis or early treatment of acute pulmonary diseases in mammals, and the pharmaceutical preparation being a pulmonary surfactant preparation. The secondary packaging, the primary packaging comprising the pharmaceutical preparation and the pack insert otherwise correspond to what the person skilled in the art would regard as standard for pharmaceutical preparations of this type. Suitable primary packagings are, for example, ampoules or bottles of suitable materials such as transparent polyethylene or glass or alternatively suitable means of administration such as are customarily employed for the administration of active compounds into the lungs. By way of example, mention may be made of means of administration for the atomization of an active compound solution or suspension or for the atomization of active compound powder. Preferably, the primary packaging is a glass bottle which can be sealed, for example, by a commercially available rubber stopper or a septum. A suitable secondary packaging which may be mentioned by way of example is a folding box.

If desired, the pulmonary surfactant preparations can also be administered in combination with other medicaments in the prophylaxis or early treatment of acute lung diseases, in particular with those medicaments which are customarily employed for the treatment of acute lung diseases.

### EXAMPLES

A.) Production of Powdered Pulmonary Surfactant Preparations

Powdered pulmonary surfactant preparations are produced by the process described in WO 97/26863:

### Example 1

7.0 g of 1,2-dipalmitoyl-3-sn-phosphatidylcholine, 2.5 g of 1-palmitoyl-2-oleoyl-3-sn-phosphatidylglycerol sodium, 205 mg of calcium chloride dehydrate and 250 mg of palmitic acid are dissolved in 300 ml of ethanol/water (85:15) with warming to 60° C., cooled to room temperature and mixed with 350 ml of a solution of rSP-C (FF/I) In chloroform/methanol 9:1 (c=429 mg/l). The resulting solution is spray-dried in a Büchi B 191 laboratory spray dryer. Spray conditions: drying gas air, inlet temperature 90° C., outlet temperature 52-54° C. A relatively loose powder is obtained.

### Example 2

A solution of the surfactant obtained from bovine lungs (obtained by extraction and purification steps such as described, for example, in EP 406732) in chloroform/methanol is spray-dried under the following conditions: Büchi B 191 laboratory spray dryer, drying gas nitrogen, inlet temperature 80° C., outlet temperature 50-52° C. A fine, yellowish powder is obtained.

### Example 3

10.95 g of 1,2-dipalmitoyl-3-sn-phosphatidylcholine, 4.6 g of 1-palmitoyl-2-oleoyl-3-sn-phosphatidylglycerol ammonium, 418 mg of calcium chloride dihydrate and 750 mg of palmitic acid are dissolved in 330 ml of 2-propanol/water (85:15) at 50° C. and, after cooling to 30° C., mixed with 620 ml of a solution of rSP-C (FF/I) in isopropanol/water (95:5, c=484 mg/l). The resulting solution is spray-dried in a Büchi B 191 laboratory spray dryer. Spray conditions: drying gas nitrogen, inlet temperature 100° C., outlet temperature 58-60° C. A colorless powder is obtained.

### Example 4

3.74 g (5.1 mmol) of 1,2-dipalmitoyl-3-sn-phosphatidylcholine, 2.81 g (3.7 mmol) of 1-palmitoyl-2-oleoyl-3-sn-phosphatidylcholine, 2.90 g (3.9 mmol) of 1,2-dipalmitoylphosphatidyl-3-sn-phosphatidylglycerol sodium, 234 mg of palmitic acid and 279 mg (1.9 mmol) of calcium chloride dihydrate are dissolved in 160 ml of 2-propanol/water (85: 15) at 50° C. and, after cooling to 30° C., mixed with 566 ml of a solution of rSP-C (FF/I) in isopropanol/water (92:8, c=330 mg/l) at 30° C. The resulting solution is spray-dried in a Büchi B 191 laboratory spray dryer. Spray conditions: drying gas nitrogen, inlet temperature 90° C., outlet temperature 58-60° C. A colorless powder is obtained.

### Example 5

0.5 g of KL4 (INN: sinapultide), 7.125 g of 1,2-dipalmitoyl-3-sn-phosphatidylcholine and 2.43 g of 1-palmitoyl-2-oleoyl-3-sn-phosphatidylglycerol ammonium are dissolved in 500 ml of chloroform/methanol 1:1 with warming to 45° C. and then spray-dried in a Büchi B 191 laboratory spray dryer. Spray conditions: drying gas nitrogen, inlet temperature 85° C., outlet temperature 55° C. A colorless powder is obtained.

### Example 6

A solution of phospholipids, palmitic acid and calcium chloride dihydrate obtainable according to Example 1, 3 or 4 is spray-dried—without addition of a solution of rSP-C (FF/I)—corresponding to the conditions according to Example 1, 3 or 4. A powder is obtained.

B.) Production of the Medicaments According to the Invention

### Example 1

0.1 to 10 g of the powder obtained according to Example 1 are dispensed into a bottle of volume 100 to 250 ml and the bottle is sealed. The bottle is packed in a suitable folding box together with a pack insert.

1. The use of a pulmonary surfactant preparation for the production of medicaments for the prophylaxis or early treatment of acute pulmonary diseases in mammals.

2. The use as claimed in claim 1, the mammals being human patients in which the risk of ARDS or ALI exists, patients in which the risk of acute respiratory insufficiency exists, patients in which the risk of pneumonia exists, patients in which the risk of a nosocomial infection exists, patients with hypothermia or patients with SIRS (systemic inflammatory response syndrome) associated with ALI.

3. The use as claimed in claim 2, patients being selected from the following patient groups: patients before, during or after an intervention on the open thorax, patients who are ventilated, patients with pulmonary intoxication, patients with a trauma, patients with sepsis, patients with pneumonia or those in which the risk of pneumonia exists, patients with a nosocomial infection or in which the risk of a nosocomial infection exists, patients with hypothermia and patients with SIRS (systemic inflammatory response syndrome) associated with ALI.

4. The use as claimed in claim 2, the pulmonary surfactant preparation comprising phospholipids, the pulmonary surfactant proteins SP-B and/or SP-C and/or their modified derivatives, if desired together with further excipients.

5. The use as claimed in claim 4, the pulmonary surfactant protein being recombinantly prepared pulmonary surfactant proteins.

6. The use as claimed in claim 4, the pulmonary surfactant protein being lusupultide.

7. The use as claimed in claim 3, the patients being selected from the following group: patients before, during or after a heart operation, patients in which an intervention on the lung is carried out, patients having respiratory insufficiency, patients having central or peripheral respiratory paralysis, patients having ventilation under anesthesia, patients having long-term ventilation in intensive medicine, patients having pulmonary intoxication as a result of a bone marrow transplantation (toxic lung injury after bone marrow transplantation), patients having a pulmonary intoxication which was caused by toxic gases, patients having thoracic, cranial or cerebral trauma or those having multiple traumas, and patients having a lowered body temperature in the case of collapse, hypothyroidism, cachexia, accidental hypothermia due to exposure to cold and controlled hypothermia.

8. A process for the prophylaxis or early treatment of acute pulmonary diseases in mammals; including humans, a therapeutically efficacious and pharmacologically tolerable amount of a pulmonary surfactant preparation being administered to the sick mammal.

9. A commercial product, comprising a customary secondary packaging, a primary packaging comprising a pharmaceutical preparation and, if desired, a pack insert, the pharmaceutical preparation being suitable for the prophylaxis or early treatment of acute pulmonary diseases in mammals and reference being made on the secondary packaging or on the pack insert of the commercial product to the suitability of the pharmaceutical preparation for the prophylaxis or early treatment of acute pulmonary diseases in mammals, and the pharmaceutical preparation being a pulmonary surfactant preparation.
